(19) United States
(12) Patent Application Publication
(10) Pub. No.: US 2010/0094070 A1
(43) Pub. Date: Apr. 15, 2010

(54) ETHYLENE TRIMERIZATION USING A SUPPORTED CHROMIUM-TANTALUM CATALYST

(75) Inventors: He Qiu, Trenton, NJ (US); Bing Zhou, Cranbury, NJ (US)

Correspondence Address: Workman Nydegger;1000 Eagle Gate Tower, 60 East South Temple, Salt Lake City, UT, 84111, US

(73) Assignee: HEADWATERS TECHNOLOGY INNOVATION, LLC, Lawrenceville, NJ (US)

(21) Appl. No.: 12/252,179

(22) Filed: Oct. 15, 2008

(51) Int. Cl. C07C 2/24; B01J 23/20; B01J 23/26; B01J 21/08; B01J 21/04; B01J 29/04; B01J 27/132; B01J 27/25; B01J 27/053; B01J 27/188; B01J 31/12

(52) U.S. Cl. 585/513; 502/311; 502/246; 502/87; 502/182; 502/228; 502/201; 502/217; 502/210; 502/161; 502/154; 502/200

(57) ABSTRACT

Bimetallic, supported catalysts for production of 1-hexene from ethylene are manufactured by impregnating a porous, solid support material with at least one catalytic chromium compound and at least one catalytic tantalum compound. The bimetallic, supported catalysts have high catalytic turnover, high selectivity for 1-hexene production, a low tendency for metals to leach from the catalysts during manufacturing and use compared to catalysts manufactured using known techniques. Moreover, the catalysts can be reused in multiple synthesis runs. High turnover, high selectivity, and reusability improve yields and reduce the costs associated with producing 1-hexene from ethylene, while the absence of metal leaching reduces the potential environmental impacts of using toxic metal catalysts (e.g., chromium).

### BACKGROUND OF THE INVENTION

1. The Field of the Invention

The present invention relates generally to a supported, bimetallic catalyst for the production of 1-hexene from ethylene.

2. The Relevant Technology

Transition metal catalysts play a very important role in numerous industrial chemical processes, including pharmaceuticals manufacturing, petroleum refining, and chemical synthesis, among others. Cost pressures and the need for improved synthesis routes have led to continued improvement in catalyst performance. The catalytic production of 1-hexene from ethylene monomer is of particular interest.

The selective trimerization of ethylene to prepare primarily 1-hexene, and ultimately to form polymers therefrom, has been extensively studied and a number of catalysts developed. Linear alpha olefins such as 1-hexene are widely used in the chemical industry. There is considerable industrial interest in the selective trimerization of ethylene to 1-hexene.

The general mechanism of metal catalyzed ethylene trimerization involves formation of a metallocycle that results from the joining of the catalytic metal and three ethylene monomers. The active catalytic metal species is regenerated with the release of 1-hexene through an intermolecular hydride transfer reaction. Further discussion of the mechanism of metal catalyzed ethylene trimerization can be found in Angew. Chem. Int. Ed., 42, (2003), 808-810, the entirety of which is incorporated herein by specific reference.

Examples of ethylene trimerization processes for the production of 1-hexene include the well known chromium pyrrolide complexes, disclosed in U.S. Pat. Nos. 5,523,507, 5,786,431, and elsewhere; trialkylsilylamide-chromium (II) complexes on activated inorganic refractory compounds in combination with aluminum triallyl compounds, disclosed in U.S. Pat. No. 5,104,841; chromium diphosphines, disclosed in Chem. Comm. (2002) p 858; chromium cyclopentadienyl catalysts as disclosed in Angew. Chem. Int. Ed. 38 (1999), p 428, J. Poly. Sci., 10 (1972), p 2621, and Applied Catalysis A; General 255, (2003), p 355-359; silica supported trialkylsilylamide-chromium complexes in combination with isobutylalumoxane, disclosed in J. Mol. Cat. A: Chemical, 187, (2002), p 135-141; mixed heteroatomic compounds disclosed in Chem. Comm. (2003), p 334; titanium cyclopentadiene catalysts such as those of Angew. Chem. Int. Ed., 40, (2001), p 2516; and numerous others. In U.S. Pat. No. 5,137,994, a process for producing ethylene/1-hexene copolymers directly from ethylene using silica supported chromium compounds was disclosed. Control of polymer density was obtained by adjusting the ethylene/1-hexene ratio of the intermediate monomer mixture obtained in an initial trimerization.

Important performance characteristics to be considered in choosing a catalytic system for the trimerization of ethylene include catalyst activity (i.e., catalytic turnover rate), reaction selectivity, and relative catalyst cost. Many catalysts are capable of catalyzing the reaction of ethylene monomers to produce a variety of products. But many catalysts suffer from low turnover, low selectivity for 1-hexene production, or high cost.

Many ethylene trimerization catalysts are organometallic compounds that are soluble in the ethylene trimerization reaction mixture. While many soluble organometallic catalysts exhibit impressive turnover rates and selectivity, they typically leave metal contaminants that must be removed before the reaction products are usable. Moreover, such catalysts are typically not reusable and their disposal presents difficulties because many catalytic metals can be hazardous if they are released into the environment.

### BRIEF SUMMARY OF THE INVENTION

The present invention pertains to a bimetallic, supported catalyst for production of 1-hexene from ethylene. The bimetallic, supported catalyst has a high turnover rate and high selectivity for 1-hexene production. Surprisingly, the catalyst can be reused multiple times without significant loss of turnover rate or selectivity, the catalyst retains selectivity for 1-hexene production ad high turnover even after being exposed to air, and the catalytic metals do not show a tendency to leach out of the porous, solid support material even when the catalyst is reused multiple times. High turnover, high selectivity, air stability, and reusability improve yields and reduce the costs associated with producing 1-hexene from ethylene, while the absence of metal leaching reduces the potential environmental impacts of using toxic metal catalysts (e.g., chromium).

In one embodiment, a bimetallic, supported catalyst for trimerization of ethylene is provided. The catalyst includes a porous, solid support material, at least one catalytic chromium compound disposed on the porous, solid support material, and at least one catalytic tantalum compound disposed on the porous, solid support material.

In one embodiment, the porous, solid support material includes at least one of silica, alumina, zeolite, activated carbon, a molecular sieve such as ZSM-5 or CMF-41, and combinations thereof. In a preferred embodiment, the porous, solid support material is silica. The silica employed in the catalyst is preferably pure but may contain minor amounts of other inorganic oxides such as alumina, titania, zirconia, magnesia and the like.

In one embodiment, either the catalytic chromium compound or the catalytic tantalum compound or both are chemically bonded to the porous, solid support material. In one embodiment, the chemical bond to the support material includes, but is not limited to, a metal-oxygen bond via an oxygen that is itself bonded to the support material.

In one embodiment, the catalytic chromium compound of the catalyst includes at least one Cr(III) species (i.e., the oxidation state of the Cr is 3+) bound to a suitable number of ligands. Suitable ligands for the Cr(III) species include oxygen, chloride, bromide, fluoride, nitrate, sulfate, phosphate, acetate, acetylacetonate, 2-ethyl hexanoate, bistrimethylsilylamido (NTMS₂), derivatives thereof, and combinations thereof. As used herein, the term “derivatives thereof” includes oxide derivatives of the catalytic chromium such as would be observed if the metal forms a metal-oxygen bond via an oxygen that is itself bonded to the support material. In such a case, one of the ligands (e.g., chloride, bromide, fluoride, nitrate, sulfate, phosphate, acetate, acetylacetonate, 2-ethyl hexanoate, or NTMS₂) would be displaced by the formation of the metal-oxygen bond in order to maintain the 3+ oxidation state of the chromium.

In one embodiment, a Cr(III) species bound to a suitable number of ligands can be schematically represented according to formula 1:

˜O—CrX₂ Formula 1

where the 3+ oxidation state of the Cr is satisfied by an oxygen used to bond the Cr to the support material and two other ligands (i.e., X₂) bonded to the Cr. For example, Formula 1 could be satisfied by a Cr(III) species with the following bonding: ˜O—Cr(NTMS₂)₂.

In one embodiment, the catalytic tantalum compound includes at least one Ta(V) species (i.e., the oxidation state of the Ta is 5+) bound to a suitable number of ligands. Suitable ligands for the Ta(V) species include oxygen, chloride, bromide, fluoride, iodide, pentamethylcyclopentadienyl chloride (i.e., TaCp*Cl₄), dimethylamine (NMe₂), dimethylamine chloride, hydrotris(pyrazolyl)borato chloride (i.e., TaTpCl₄), hydrotris(3,5-dimethylpyrazolyl)borato chloride (i.e., TaTp*Cl₄), derivatives thereof, and combinations thereof. As used herein, the term “derivatives thereof” includes oxide derivatives of the catalytic tantalum such as would be observed if the metal forms a metal-oxygen bond via an oxygen that is itself bonded to the support material. In such a case, one of the ligands (e.g., chloride, bromide, fluoride, iodide, Cp*, NMe₂, Tp, or Tp*) would be displaced by the formation of the metal-oxygen bond in order to maintain the 5+ oxidation state of the tantalum.

In one embodiment, a Ta(V) species bound to a suitable number of ligands can be schematically represented according to formula 2:

˜O—TaZ₄ Formula 2

where the 5+ oxidation state of the Ta is satisfied an oxygen used to bond the Ta to the support material and four other ligands (i.e., Z₄) bonded to the Ta. For example, Formula 2 could be satisfied by a Ta(V) species with the following bonding: ˜O—TaCp*Cl₃ (i.e., the Ta is bonded to one oxygen atom, one pentamethylcyclopentadienyl species, and three chlorine atoms).

In one embodiment, a method of making a bimetallic, supported ethylene trimerization catalyst includes the steps of: (a) preparing at least one catalytic chromium(III) (Cr(III)) compound by reacting at least one halogenated chromium compound with at least one organometallic reagent, (b) preparing a precursor solution that includes the at least one catalytic chromium compound as prepared in step (a) and at least one catalytic tantalum(V) (Ta(V)) compound (e.g., TaCl₅, TaCp*Cl₄, and Ta(NMe₂)₅, which can be purchased from Strem Chemicals, Inc.), in which the molar ratio of chromium to tantalum is in a range of about 15:1 to about 1:1, (c) impregnating a porous, solid support material with the precursor solution to yield a bimetallic, supported catalyst for trimerization of ethylene having at least one Cr(III) catalytic metal and at least one Ta(V) catalytic metal. All of the steps involved in preparing catalyst are preferably performed under a dry argon atmosphere with the use of either a dry box or standard Schlenk techniques.

High surface area silica that is appropriate for preparing the catalyst can be purchased from Saint-Gobain Norpro. In one embodiment, the silica support material may be pretreated prior to impregnation with the catalytic metals by calcining at a temperature in a range from about 100° C. to about 500° C. for about 10 minutes to about 5 hours.

In one embodiment, the method further includes the steps of (1) washing the impregnated porous, solid support material with at least one solvent to remove unbound chromium and tantalum species, and (2) removing the solvent to yield a cleaned bimetallic, supported catalyst for trimerization of ethylene. Suitable examples of techniques for removing residual cleaning solvent left on the supported catalyst after filtration include, but are not limited to, evaporating the solvent under vacuum.

In one embodiment, the chromium:tantalum ratio in the finished bimetallic, supported ethylene trimerization catalyst is in a range from about 5:1 to about 1:5. Preferably, the chromium:tantalum ratio in the finished bimetallic, supported ethylene trimerization catalyst is in a range from about 2:1 to about 1:2.

In one embodiment, a method for catalytically producing 1-hexene from ethylene includes the steps of (a) providing a bimetallic, supported catalyst that includes chromium and tantalum, (b) forming a reaction mixture in a reaction vessel, the reaction mixture including the bimetallic, supported catalyst, an organic solvent, pressurized ethylene gas, and a trialkyl-aluminum compound, 2,5-dimethylpyrrole, and hexachloroethane in amounts sufficient for catalysis, (c) reacting the reaction mixture in the reaction vessel to yield 1-hexene.

In one embodiment, the reaction temperature in the reaction vessel is in a range between about 50° C. and about 140° C. Preferably, the reaction temperature is in a range between 70° C. and about 125° C., more preferably in a range between about 90° C. and about 110° C.

In one embodiment, the pressure of the ethylene gas in the reaction vessel is in a range from about 1 bar to about 100 bar. Preferably, the ethylene gas pressure is in a range from about 25 bar to about 85 bar, more preferably in a range between about 50 bar to about 70 bar.

In one embodiment, the catalytic turnover (i.e., the rate of conversion of ethylene to 1-hexene) is in a range from about 100 g 1-hexene/g metal/hr to about 5200 g hexene/g metal/hr, preferably with a selectivity for 1-hexene of at least 50%, or more preferably at least 70%, or most preferably at least 90%.

These and other advantages and features of the present invention will become more fully apparent from the following description and appended claims, or may be learned by the practice of the invention as set forth hereinafter.

### DETAILED DESCRIPTION OF THE PREFERRED EMBODIMENTS

### I. Introduction

The present invention pertains to a bimetallic, supported catalytic system for production of 1-hexene from ethylene. In particular, the present invention pertains to a bimetallic, supported catalyst that includes at least one catalytic chromium compound and at least one catalytic tantalum compound disposed on a porous, solid support material. The bimetallic, supported catalyst has a high turnover rate and high selectivity for 1-hexene production. Surprisingly, the catalyst can be reused multiple times without significant loss of turnover rate or selectivity, the catalyst retains selectivity for 1-hexene production ad high turnover even after being exposed to air, and the catalytic metals do not show a tendency to leach out of the porous, solid support material even when the catalyst is reused multiple times. High turnover, high selectivity, air stability, and reusability improve yields and reduce the costs associated with producing 1-hexene from ethylene, while the absence of metal leaching reduces the potential environmental impacts of using toxic metal catalysts (e.g., chromium).

### II. Components Used to Manufacture a Bimetallic, Supported Ethylene Trimerization Catalyst

The following components can be used to carry out the steps for manufacturing a bimetallic, supported ethylene trimerization catalyst according to the present invention.

A. Catalytic Metals

As mentioned above, the bimetallic, supported catalyst disclosed herein has a high turnover rate and high selectivity for 1-hexene production, the catalyst can be used in multiple 1-hexene synthesis reactions, and the catalytic metals do not leach out of the support material even when the catalyst is reused multiple times.

In one embodiment, the metals used to form the supported, bimetallic catalyst can include at least one chromium compound and at least one tantalum compound. As such, the metals have an oxidation state that is above their ground state. In a preferred embodiment, the chromium has an oxidation state of 3+ (i.e., Cr(III)) and the tantalum has an oxidation state of 5+ (i.e., Ta(V)). One will appreciate, however, that the at least one chromium compound and/or at least one tantalum compound can have an oxidation state that is higher or lower that Cr(III) and/or Ta(V).

In one embodiment, the chromium:tantalum ratio in the catalyst of the present invention is in a range between about 5:1 to 1:5. Preferably, the chromium:tantalum ratio in the catalyst is in a range between about 3:1 or 1:3, or more preferably in a range between about 2:1 to 1:2.

B. Ligands

Metal binding ligands are selected in order to satisfy the oxidation state of the metals. In addition, ligands are selected to favor the efficient transformation of ethylene to 1-hexene. For example, the size of the ligands bound to the metals can affect specificity through steric effects. That is, space restrictions around the catalytic metal centers can affect selectivity if the free space around the metal center is favorable for 1-hexene production but not for other reaction products. Ligands can also affect reaction kinetics by making the production of 1-hexene more kinetically favorable relative to other potential reaction products.

For example, pentamethylcyclopentadienyl (Cp*), which is included in some of the catalysts described herein, is a useful organometallic ligand arising from the binding of the five ring-carbon atoms in C₅Me₅-, or Cp*-, to metals. Relative to the more common cyclopentadienyl (Cp) ligand, Cp* offers certain features that are often advantageous. Being more electron-rich, Cp* is a stronger donor and is less easily removed from the metal. Consequently its complexes exhibit increased thermal stability. Moreover, its steric bulk tends to attenuate intermolecular interactions, increasing the tendency in this instance to form 1-hexene and decreasing the tendency to form polymeric structures. Its complexes also tend to be highly soluble in non-polar solvents.

In one embodiment, the catalytic chromium compound of the catalyst includes at least one Cr(III) species (i.e., the oxidation state of the Cr is 3+) bound to a suitable number of ligands. Suitable ligands for the Cr(III) species include oxygen, chloride, bromide, fluoride, nitrate, sulfate, phosphate, acetate, acetylacetonate, 2-ethyl hexanoate, bistrimethylsilylamido (NTMS₂), derivatives thereof, and combinations thereof. As used herein, the term “derivatives thereof” includes oxide derivatives of the catalytic chromium such as would be observed if the metal forms a metal-oxygen bond via an oxygen that is itself bonded to the support material. In such a case, one of the ligands (e.g., chloride, bromide, fluoride, nitrate, sulfate, phosphate, acetate, acetylacetonate, 2-ethyl hexanoate, or NTMS₂) would be displaced by the formation of the metal-oxygen bond in order to maintain the 3+ oxidation state of the chromium.

In one embodiment, a Cr(III) species bound to a suitable number of ligands can be schematically represented according to formula 1:

˜O—CrX₂ Formula 1

where the 3+ oxidation state of the Cr is satisfied by an oxygen used to bond the Cr to the support material and two other ligands (i.e., X₂) bonded to the Cr. For example, Formula 1 could be satisfied by a Cr(III) species with the following bonding: ˜O—Cr(NTMS₂)₂.

In one embodiment, the catalytic tantalum compound includes at least one Ta(V) species (i.e., the oxidation state of the Ta is 5+) bound to a suitable number of ligands. Suitable ligands for the Ta(V) species include oxygen, chloride, bromide, fluoride, iodide, pentamethylcyclopentadienyl chloride (i.e., TaCp*Cl₄), dimethylamine (NMe₂), dimethylamine chloride, hydrotris(pyrazolyl)borato chloride (i.e., TaTpCl₄), hydrotris(3,5-dimethylpyrazolyl)borato chloride (i.e., TaTp*Cl₄), derivatives thereof, and combinations thereof. As used herein, the term “derivatives thereof” includes oxide derivatives of the catalytic tantalum such as would be observed if the metal forms a metal-oxygen bond via an oxygen that is itself bonded to the support material. In such a case, one of the ligands (e.g., chloride, bromide, fluoride, iodide, Cp*, NMe₂, Tp, or Tp*) would be displaced by the formation of the metal-oxygen bond in order to maintain the 5+ oxidation state of the tantalum.

In one embodiment, a Ta(V) species bound to a suitable number of ligands can be schematically represented according to formula 2:

˜O—TaZ₄ Formula 2

where the 5+ oxidation state of the Ta is satisfied an oxygen used to bond the Ta to the support material and four other ligands (i.e., Z₄) bonded to the Ta. For example, Formula 2 could be satisfied by a Ta(V) species with the following bonding: ˜O—TaCp*Cl₃ (i.e., the Ta is bonded to one oxygen atom, one pentamethylcyclopentadienyl species, and three chlorine atoms).

C. Support Materials

The support materials are highly porous inorganic materials. The support material is typically selected to have a particular composition, particle size and shape, surface area, and initial crush strength. Suitable support materials include at least one of silica, alumina, zeolite, activated carbon, or least one molecular sieve such as ZSM-5 or CMF-41, and combinations thereof. In a preferred embodiment, the porous, solid support material is silica. The silica employed in the catalyst is preferably pure but may contain minor amounts of other inorganic oxides such as alumina, titania, zirconia, magnesia and the like.

The shape of the support material can affect the performance of the catalyst during use. In one embodiment, the shapes used in the invention are particulates. Examples of suitable particulate structures include spheres, cylinders, pellets, beads, rings, trilobes, stars, and the like. In one embodiment the particulate support materials have an average particle size of less than 100 mm, more preferably less than 50 mm and most preferably less than 10 mm. Alternatively, the particulate support has a diameter in a range from about 100 nm to about 20 mm, alternatively from about 1 mm to about 10 mm, or in yet another embodiment from about 1 mm to about 5 mm. The surface area of the support can range from about 1 m²/g to about 2,000 m²/g, more preferably from about 20 m²/g to about 500 m²/g.

In one embodiment, either the catalytic chromium compound or the catalytic tantalum compound or both are chemically bonded to the porous, solid support material. In one embodiment, the chemical bond to the support material includes, but is not limited to, a metal-oxygen bond via an oxygen that is itself bonded to the support material.

### III. Methods of Making Supported Catalyst

Example methods for manufacturing supported, bimetallic catalyst according to the invention can be broadly summarized as follows. First, one or more catalytic chromium catalytic chromium(III) (Cr(III)) compounds are prepared by reacting at least one halogenated chromium compound with at least one organometallic reagent. For example, CrCl₃ may be reacted with Li(NTMS₂) to produce a mixture that includes the catalytic Cr(III) compounds CrCl₃ and Cr(NTMS₂)₃. Second, a precursor solution is prepared that includes the at least one catalytic chromium compound that is prepared in the first step and at least one catalytic tantalum(V) (Ta(V)) compound, in which the molar ratio of chromium to tantalum is preferably in a range of about 15:1 to about 1:1, more preferably about 13:1 to about 2:1, most preferably about 12:1 to about 4:1. Third, the supported, bimetallic catalyst is formed by impregnating a porous, solid support material with the precursor solution containing the at least one Cr(III) catalytic metal compound and at least one Ta(V) catalytic metal compound. Generally, an excess of the Cr species is used in the precursor solution that is used to impregnate the support material because the Cr species are generally less reactive toward the support material relative to the Ta species.

Suitable support materials include at least one of silica, alumina, zeolite, activated carbon, or least one molecular sieve such as ZSM-5 or CMF-41, and combinations thereof. In a preferred embodiment, the porous, solid support material is silica.

The support material is typically selected to have a sufficient surface area for supporting the desired loading and type of chromium and tantalum compounds. In addition, the support is selected to have a size and shape that is suitable for the particular application that the supported catalyst will be used in (e.g., a fixed bed reactor for synthesizing 1-hexene). Those skilled in the art are familiar with selecting porous support materials to provide a proper metal loading, size, and shape for various reactions and reactor configurations. The silica support material may be pretreated prior to impregnation with the catalytic metals by calcining at a temperature in a range from about 100° C. to about 500° C. for about 10 minutes to about 5 hours.

In one embodiment, the impregnating process includes the formation of chemical bonds between the porous, solid support material and the chromium and tantalum compounds. In one embodiment, the bonds are metal-oxygen bonds between oxygen atoms that are bound to the support material. The metal-oxygen bond is a very strong bond and, as such, the chromium and tantalum compounds can be tightly bound to the porous, solid support material. One consequence of the bonding between the support material and the chromium compounds and/or the tantalum compounds is that the metals do not show a tendency to leach out of the catalyst during the trimerization reaction. This makes it easy to separate the catalyst from the reaction products and it alleviates many of the environmental hazards associated with homogenous catalysts.

In one embodiment, the method of preparing the supported, bimetallic catalyst can further includes steps of (1) washing the impregnated porous, solid support material with at least one solvent to remove unbound chromium and tantalum species, and (2) removing the solvent. Suitable examples of techniques for removing the solvent include, but are not limited to, evaporating the solvent under vacuum

The final catalyst typically includes chromium and tantalum in a ratio of about 5:1 to about 1:5. Preferably, the chromium:tantalum ratio in the finished bimetallic, supported ethylene trimerization catalyst is in a range from about 2:1 to about 1:2.

### IV. Methods of Manufacturing 1-Hexene

The supported catalysts of the present invention are particularly advantageous for the synthesis of 1-hexene via the trimerization of ethylene monomer. This is due in part to the high selectivity of the catalysts, high catalytic turnover, low tendency to leach metals into the reaction mixture, and their reusability. In a preferred embodiment, the ethylene trimerization catalyst manufactured according to the present invention includes a combination of catalytic chromium and tantalum compounds.

The catalysts of the present invention can be used in any type of reactor suitable for the production of 1-hexene via the trimerization of ethylene. Because ethylene is a gas, the reactor typically needs to be pressurizable. Suitable reactors include fixed bed, ebullated bed, and slurry reactors. In a preferred embodiment, the catalysts of the present invention are loaded into a fixed bed or ebullated bed reactor for 1-hexene production. The use of the catalysts of the present invention in a fixed bed or ebullated bed reactor facilitates the recovery and regeneration of the catalyst.

To load the catalysts in a fixed bed or ebullated bed reactor, the supported catalysts are manufactured to have a size and/or shape suitable for a fixed bed or ebullated bed. For example, the supported catalysts can be manufactured into particulates such as beads or spheres that have a size suitable for use in a fixed bed or fluidized bed reactor. In an exemplary embodiment, the particulate has a nominal dimension of at least about 0.5 mm, and more preferably at least about 1 mm. Alternatively, the support material can be extruded to make a part with dimensions that are suitable for use in any size or shaped fixed bed reactor.

Once the supported catalyst is placed into a suitable reactor, 1-hexene can be directly synthesized by introducing reaction mixture into the reactor. The reaction mixture includes at least one organic solvent, pressurized ethylene gas, and a trialkyl-aluminum compound (e.g., trimethyl aluminum or triethyl aluminum), 2,5-dimethylpyrrole, and hexachloroethane in amounts sufficient for catalysis. The reaction mixture is reacted at a controlled temperature and pressure for a period of time in order to yield 1-hexene.

In one embodiment, the reaction temperature in the reaction vessel is maintained in a range between about 50° C. and about 140° C. Preferably, the reaction temperature is in a range between about 90° C. and about 110° C.

In one embodiment, the pressure of the ethylene gas in the reaction vessel is in a range from about 1 bar to about 100 bar. Preferably, the ethylene gas pressure is in a range from about 50 bar to about 70 bar.

### V. Examples

The following examples are exemplary procedures for manufacturing bimetallic, supported ethylene trimerization catalysts according to the invention and for manufacturing 1-hexene using these catalysts.

### Example 1

### Catalyst Preparation

Example 1 describes a method for preparing a chromium-tantalum supported on silica. A supported ethylene trimerization catalyst was prepared using the following protocol: First, CrCl₃ (2.01 g, 12.7 mmol) and Li(NTMS₂) (6.31 g, 37.8 mmol) were mixed in tetrahydrofuran (THF) (20 mL) and stirred at room temperature for 4 hr. A green solution was observed. Second, 0.5 ml of the Cr/THF solution was combined with TaCl₅, TaCp*Cl₄, or Ta(NMe₂)₅ (0.025 mmol to 0.075 mmol) in 20 ml of heptane. The solution was allowed to impregnate SiO₂ (2 g) beads for 2 hr. Excess solvent and chromium and tantalum were removed from the beads by filtration. Heptane was then used to wash the catalyst until the filtrate became colorless. Residual volatiles in the catalyst were removed under vacuum to give light blue catalyst beads.

All manipulations were performed under a dry argon atmosphere with the use of either a dry box or standard Schlenk techniques. TaCl₅, TaCp*Cl₄, and Ta(NMe₂)₅ were purchased from Strem Chemicals, Inc. High surface area silica (surface area=179 m²/g, pore volume=0.74 cm³/g) was purchased from Saint-Gobain Norpro.

### Example 2

### Catalyst Preparation

Example 2 describes another method for preparing a chromium-tantalum supported on silica. A supported ethylene trimerization catalyst was prepared using a protocol similar to Example 1 except the silica beads were calcined at 450° C. prior to being impregnated with the chromium-tantalum solution.

### Example 3

### General Procedure for Ethylene Trimerization

Example 3 describes a general procedure for ethylene trimerization using the catalysts of the present invention. Catalysts were prepared as described above. Under an inert atmosphere (dry argon), catalyst (50 mg) was mixed in 10 mL of heptane in a glass autoclave liner equipped with a Teflon-coated stir bar. After AlEt₃ (0.05 ml, 1 M in heptane), 2,5-dimethylpyrrole (0.005 ml) and hexachloroethane (1.7 mg) were added to the solution, the 75 ml stainless autoclave was removed from the drybox. The autoclave was charged with 4.8 MPa (48 bar) of ethylene, and the mixture was stirred at 100° C. for 2 hr. The autoclave was then chilled in ice for 30 min and vented of ethylene. After filtration, the yield and selectivity were detected by gas chromatographic (GC) analysis.

### Example 4

### The Effect of Catalyst Composition

Example 4 describes the effect of catalyst composition on turnover and selectivity for 1-hexene production. In particular, Example 4 describes the effect of different including different tantalum compounds in the catalyst. Catalysts were prepared as described above in Example 2. In Samples 26, 28, and 29 the Cr impregnated into the silica beads is assumed to be a mixture of CrCl₃ and Cr(NTMS₂)₃. In addition, the active form of the metals in the catalysts may be different than what is shown. For example, one or more ligands associated with the chromium or tantalum compounds may be displaced by the formation of one or more bonds between the metal and the silica support material. Nevertheless, the oxidation states of the catalytically active metals are Cr(III) and Ta(V).

Experimental results for different tantalum compounds are shown in table 1. The results suggest that all the tantalum compounds and chromium mixture have the high selectivity from 85 to 90%. In terms of activity, Ta(NMe₂)₅ and chromium compounds (i.e., Sample-26) provide the highest activity yielding 5150 g of 1-hexene/(g M h).

TABLE 1

| | | | | | turnover (g 1- | Selectivity |
|---|---|---|---|---|---|---|
| | ALEt₃ | Cl₃CCCL₃ | Pyrrole | T (° C.) | hexene/g M/h) | (% from area) |
| Sample-26 | 0.05 ml | 0.002 | 0.005 ml | 100 | 5150 | 90.2 |
| Sample-28 | 0.05 ml | 0.002 | 0.0025 ml | 100 | 1388 | 88.6 |
| Sample-29 | 0.05 ml | 0.002 | 0.005 ml | 100 | 1011 | 86.7 |

Sample-26 (Silica/Cr/Ta(NMe₂)₅) (Cr 0.14%, Ta 0.09%)
Sample-28 (Silica/Cr/TaCl₅) (Cr 0.21%, Ta 0.17%)
Sample-29 (Silica/Cr/TaCp*Cl₄) (Cr 0.40, Ta 0.65%)

### Example 5

### Effect of Temperature and Pressure

There are many factors that contribute to the efficiency of the catalytic process. Some of the factors that contribute to the overall efficiency are temperature, pressure, and catalyst composition. Example 5 describes the effect of temperature and pressure on catalytic turnover and selectivity.

The general procedure as described in Example 3 was applied, except the catalyst components, temperature, and pressure were changed. Larger amount of AlEt₃, 2,5-dimethylpyrrole and hexachloroethane have been used to run the reaction. In each run, 10 ml heptane was used as a solvent. In Samples 18, 20, 21, and 22 the Cr impregnated into the silica beads is assumed to be a mixture of CrCl₃ and Cr(NTMS₂)₃. In addition, the active form of the metals in the catalysts may be different than what is shown. For example, one or more ligands associated with the chromium or tantalum compounds may be displaced by the formation of one or more bonds between the metal and the silica support material. Nevertheless, the oxidation states of the catalytically active metals are Cr(III) and Ta(V).

A total of six pressures and five temperatures (70, 65, 60, 50, 40 and 30 bar & 140, 120, 100, 75 and 50° C.) were used to conduct the studies with catalyst Samples 18, 20, 21 and 22. The turnover numbers and selectivity results with different temperature and pressure are listed in Tables 2 and 3.

TABLE 2

| | | | | | turnover (g hexene/ | Selectivity (% from |
|---|---|---|---|---|---|---|
| | ALEt₃ | Cl₃CCCL₃ | Pyrrole | T (° C.) | g Cr per h) | area) |
| Sample-18 | 2.0 ml | 0.05 | 0.02 ml | 140 | 444 | 30.2 |
| Sample-18 | 2.0 ml | 0.05 | 0.02 ml | 120 | 768 | 44.1 |
| Sample-18 | 2.0 ml | 0.05 | 0.02 ml | 100 | 807 | 62.6 |
| Sample-18 | 2.0 ml | 0.05 | 0.02 ml | 75 | 718 | 61.9 |
| Sample-18 | 2.0 ml | 0.05 | 0.02 ml | 50 | 640 | 71.5 |
| Sample-20 | 2.0 ml | 0.05 | 0.02 ml | 140 | 486 | — |
| Sample-20 | 2.0 ml | 0.05 | 0.02 ml | 120 | 704 | 47.1 |
| Sample-20 | 2.0 ml | 0.05 | 0.02 ml | 100 | 893 | 69.4 |
| Sample-20 | 2.0 ml | 0.05 | 0.02 ml | 75 | 850 | 67.3 |
| Sample-20 | 2.0 ml | 0.05 | 0.02 ml | 50 | 388 | 69.7 |

Sample-18 (Silica/Cr/TaCl₅) (Cr 0.21%, Ta 0.22%)
Sample-20 (Silica/Cr/TaCp*Cl₄) (Cr 0.28, Ta 0.22%)

TABLE 3

| | | | | pressure | T | turnover (g hexene/ | Selectivity (% |
|---|---|---|---|---|---|---|---|
| | ALEt₃ | Cl₃CCCL₃ | Pyrrole | (bar) | (° C.) | g M per h) | from area) |
| Sample-21 | 2.0 ml | 0.05 | 0.02 ml | 70 | 100 | 3462 | 89.3 |
| Sample-21 | 2.0 ml | 0.05 | 0.02 ml | 65 | 100 | 2648 | 88.0 |
| Sample-21 | 2.0 ml | 0.05 | 0.02 ml | 60 | 100 | 2544 | |
| Sample-21 | 2.0 ml | 0.05 | 0.02 ml | 50 | 100 | 1071 | 77.6 |
| Sample-21 | 2.0 ml | 0.05 | 0.02 ml | 40 | 100 | 1248 | |
| Sample-21 | 2.0 ml | 0.05 | 0.02 ml | 30 | 100 | 705 | |
| Sample-22 | 2.0 ml | 0.05 | 0.02 ml | 70 | 100 | 2541 | 89.1 |
| Sample-22 | 2.0 ml | 0.05 | 0.02 ml | 65 | 100 | 3345 | 87.3 |
| Sample-22 | 2.0 ml | 0.05 | 0.02 ml | 60 | 100 | 3655 | |
| Sample-22 | 2.0 ml | 0.05 | 0.02 ml | 50 | 100 | 1678 | 78.4 |
| Sample-22 | 2.0 ml | 0.05 | 0.02 ml | 40 | 100 | 1476 | |
| Sample-22 | 2.0 ml | 0.05 | 0.02 ml | 30 | 100 | 889 | |

Sample-21 (Silica/Cr/TaCl₅) (Cr 0.21%, Ta 0.17%)
Sample-22 (Silica/Cr/TaCp*Cl₄) (Cr 0.20, Ta 0.1%)

As shown in Table 2, the highest turnover number and selectivity were obtained at 100° C. One interpretation is that there are several competing reactions in solution (e.g., polymerization of ethylene, dimerization of ethylene and trimerization of ethylene). One possible interpretation of these data is that 1-hexene formation is kinetically favored at 100° C. relative to the other temperatures that were investigated.

As shown in Table 3, the catalytic turnover and selectivity both increased with increasing pressure. The results reported here are likely due to increasing ethylene concentration in the liquid phase (i.e., the heptane solution) with an increase in pressure.

### Example 6

### The Effect of Changing AlEt₃ and Cl₃CCCl₃ Amounts

Example 6 describes the effect of changing AlEt₃ and Cl₃CCCl₃ amounts on catalytic turnover and selectivity. The general procedure was applied with changing the AlEt₃ and Cl₃CCCl₃ concentrations. The amount of AlEt₃ and Cl₃CCCl₃ were varied from 2.2 ml, 1.0 ml, 0.75 ml, 0.5 ml to 0.025 ml & from 75 mg, 50 mg, 25 mg, 13 mg to 5 mg respectively. The highest turnover number and the highest sensitivity were achieved with the solution containing 0.75 ml AlEt₃ and 25 mg Cl₃CCCl₃ (Table 4, 5).

As in previous Examples, the Cr impregnated into the silica beads is assumed to be a mixture of CrCl₃ and Cr(NTMS₂)₃. In addition, the active form of the metals in the catalysts may be different than what is shown. For example, one or more ligands associated with the chromium or tantalum compounds may be displaced by the formation of one or more bonds between the metal and the silica support material. Nevertheless, the oxidation states of the catalytically active metals are Cr(III) and Ta(V).

TABLE 4

| | | | | | turnover | |
|---|---|---|---|---|---|---|
| | | | | | (g | |
| | | | | | hexene/ | Selectivity |
| | | | | T | g M | (% |
| | ALEt₃ | Cl₃CCCL₃ | Pyrrole | (° C.) | per h) | from area) |
| Sample-18 | 2.2 ml | 0.05 | 0.02 ml | 100 | 1679 | 66 |
| Sample-18 | 1.0 ml | 0.05 | 0.02 ml | 100 | 1823 | 72 |
| Sample-18 | 0.75 ml | 0.05 | 0.02 ml | 100 | 2182 | 81 |
| Sample-18 | 0.5 ml | 0.05 | 0.02 ml | 100 | 328 | 67.9 |
| Sample-18 | 0.25 ml | 0.05 | 0.02 ml | 100 | 140 | 46.9 |
| Sample-20 | 2.2 ml | 0.05 | 0.02 ml | 100 | 1415 | 66.1 |
| Sample-20 | 1.0 ml | 0.05 | 0.02 ml | 100 | 1914 | 72.7 |
| Sample-20 | 0.75 ml | 0.05 | 0.02 ml | 100 | 1699 | 80.3 |
| Sample-20 | 0.5 ml | 0.05 | 0.02 ml | 100 | 409 | 70.5 |
| Sample-20 | 0.25 ml | 0.05 | 0.02 ml | 100 | 181 | 58.3 |

Sample-18 (Silica/Cr/TaCl₅) (Cr 0.21%, Ta 0.22%)
Sample-20 (Silica/Cr/TaCp*Cl₄) (Cr 0.28, Ta 0.22%)

TABLE 5

| | | | | | turnover | |
|---|---|---|---|---|---|---|
| | | | | | (g | |
| | | | | | hexene/ | Selectivity |
| | | | | | g M | (% |
| | ALEt₃ | Cl₃CCCL₃ | Pyrrole | T (° C.) | per h) | from area) |
| Sample-21 | 2.0 ml | 0.075 | 0.02 ml | 100 | 211 | 30.2 |
| Sample-21 | 2.0 ml | 0.05 | 0.02 ml | 100 | 1293 | 44.1 |
| Sample-21 | 2.0 ml | 0.025 | 0.02 ml | 100 | 2132 | 62.6 |
| Sample-21 | 2.0 ml | 0.013 | 0.02 ml | 100 | 1454 | 61.9 |
| Sample-21 | 2.0 ml | 0.005 | 0.02 ml | 100 | 1005 | 71.5 |
| Sample-20 | 2.0 ml | 0.075 | 0.02 ml | 100 | 208 | 58.2 |
| Sample-20 | 2.0 ml | 0.05 | 0.02 ml | 100 | 1322 | 80.2 |
| Sample-20 | 2.0 ml | 0.025 | 0.02 ml | 100 | 1814 | 70.5 |
| Sample-20 | 2.0 ml | 0.013 | 0.02 ml | 100 | 796 | |
| Sample-20 | 2.0 ml | 0.005 | 0.02 ml | 100 | 683 | |

Sample-20 (Silica/Cr(NTMS₂)₃/TaCp*Cl₄) (Cr 0.28, Ta 0.22%)
Sample-21 (Silica/Cr(NTMS₂)₃/TaCl₅) (Cr 0.21%, Ta 0.17%)

### Example 7

### Reusability of the Catalyst

Example 7 describes the effect of reusing the catalyst on catalytic turnover and selectivity. The general procedure was applied as described in Example 2 except each catalyst was used for 4 runs. Two parallel experiments for catalyst-26 are reported in Tables 6 and 7. The 2,5-dimethylpyrrole amount changes from 0.005 ml, 0.0025 ml, 0 and 0 in one set experiments (i.e., 1a-4a) 0.005 ml, 0.0025 ml, 0.0025 ml and 0 for the other set (i.e., 1b-4b).

TABLE 6

| | | | | | | turnover (g hexene/g |
|---|---|---|---|---|---|---|
| Run | ALEt₃ | Cl₃CCCL₃ | Pyrrole | | T (° C.) | M per h) |
| 1a | 0.05 ml | 0.002 | 0.005 | ml | 100 | 3334 |
| 2a | 0.05 ml | 0.002 | 0.0025 | ml | 100 | 1927 |
| 3a | 0.05 ml | 0.002 | 0 | | 100 | 472 |
| 4a | 0.05 ml | 0.002 | 0 | | 100 | 3073 |

TABLE 7

| | | | | | | turnover (g hexene/g |
|---|---|---|---|---|---|---|
| Run | ALEt₃ | Cl₃CCCL₃ | Pyrrole | | T (° C.) | M per h) |
| 1b | 0.05 ml | 0.002 | 0.005 | ml | 100 | 3331 |
| 2b | 0.05 ml | 0.002 | 0.0025 | ml | 100 | 889 |
| 3b | 0.05 ml | 0.002 | 0.0025 | ml | 100 | 2681 |
| 4b | 0.05 ml | 0.002 | 0 | | 100 | 1906 |

Sample-26 (Silica/Cr/Ta(NMe₂)₅) (Cr 0.14%, Ta 0.09%)

The experiments results suggest that at each run AlEt₃ and hexachloroethane are needed as stated in Example 3. The results are different for in terms of 2,5-dimethylpyrrole. Because of 2,5-dimethylpyrrole is a ligand, it will bond with the metal even after the solution was removed and the catalyst has been washed with pure heptane solvent. Because 2,5-dimethylpyrrole remains bound to the catalyst beads, it is likely that pyrrole is not needed from run-to-run. For example, the results (Table 6) show that the turnover numbers decrease in runs 2a and 3a due to the presence of excess pyrrole, with the turnover returning to normal in run 4a.

The results in Example 7 also demonstrate that the metals do not show a tendency to leach out of the support material even when the catalysts are used in multiple runs. That is, if the metals were leaching out, the activity would drop irreversibly. Therefore, the catalyst could be reused and metal leaching from silica support should not be an issue.

### Example 8

### Catalyst Activity Following Exposure to Oxygen

Tantalum catalysts are generally considered to be sensitive to inactivation by exposure to atmospheric oxygen. Surprisingly and unexpectedly, Example 8 demonstrates that while the catalysts of the present invention are somewhat affected by air exposure, they are not inactivated.

For the experiments, two reactions were run by the general procedure. The catalyst (Sample-26) was weighed and separately stored in two vials. One vial was kept in under argon and the second was exposed to air for 24 hr.

TABLE 8

| | | | | | turnover |
|---|---|---|---|---|---|
| | | | | | (g hexene/g |
| | ALEt₃ | Cl₃CCCL₃ | Pyrrole | T (° C.) | M per h) |
| Sample-26 | 0.05 ml | 0.002 | 0.005 ml | 100 | 3953 |
| Sample-26-air | 0.05 ml | 0.002 | 0.005 ml | 100 | 2434 |

Sample-26 (Silica/Cr/Ta(NMe₂)₅) (Cr 0.14%, Ta 0.09%)

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

We claim:

1. A bimetallic, supported catalyst for trimerization of ethylene, comprising:
a porous, solid support material;
at least one catalytic chromium compound disposed on the porous, solid support material; and
at least one catalytic tantalum compound disposed on the porous, solid support material.

2. A bimetallic, supported catalyst as recited in claim 1, wherein the porous, solid support material is selected from the group consisting of silica, alumina, zeolite, activated carbon, at least one molecular sieve, and combinations thereof.

3. A bimetallic, supported catalyst as recited in claim 1, wherein at least one of the at least one catalytic chromium compound or the at least one catalytic tantalum compound includes a chemical bond to the porous, solid support material.

4. A bimetallic, supported catalyst as recited in claim 3, wherein the bond is a metal-oxygen bond via an oxygen that is bound to the support material.

5. A bimetallic, supported catalyst as recited in claim 1, wherein the catalytic chromium compound includes at least one Cr(III) species selected form the group consisting of chromium chloride, chromium bromide, chromium fluoride, chromium nitrate, chromium sulfate, chromium phosphate, chromium acetate, chromium acetylacetonate, chromium 2-ethyl hexanoate, chromium bistrimethylsilylamido (Cr(NTMS₂)ₙ), derivatives thereof, and combinations thereof.

6. A bimetallic, supported catalyst as recited in claim 1, wherein the catalytic tantalum compound includes at least one Ta(V) species selected from the group consisting of tantalum chloride, tantalum bromide, tantalum fluoride, tantalum iodide, tantalum pentamethylcyclopentadienyl chloride (TaCp*Cl₄), tantalum dimethylamine (Ta(NMe₂)₅), Ta(NMe₂)₃Cl₂, Ta(NMe₂)₄Cl, tantalum hydrotris(pyrazolyl)borato chloride (TaTpCl₄), tantalum hydrotris(3,5-dimethylpyrazolyl)borato chloride (TaTp*Cl₄), derivatives thereof, and combinations thereof.

7. A bimetallic, supported catalyst as recited in claim 1, wherein the ratio of chromium to tantalum is in a range from about 5:1 to about 1:5.

8. A bimetallic, supported catalyst as recited in claim 1, wherein the ratio of chromium to tantalum is in a range from about 2:1 to about 1:2.

9. A reaction mixture, comprising:
the bimetallic, supported catalyst of claim 1;
an organic solvent;
pressurized ethylene gas; and
a trialkyl-aluminum compound, 2,5-dimethylpyrrole, and hexachloroethane in amounts sufficient to support catalytic conversion of ethylene to 1-hexene by the bimetallic, supported catalyst.

10. A method of making a bimetallic, supported ethylene trimerization catalyst, comprising:
(a) preparing at least one catalytic chromium(III) (Cr(III)) compound by reacting at least one halogenated chromium compound with at least one organometallic reagent;
(b) preparing a precursor solution that includes the at least one catalytic Cr(III) compound and at least one catalytic tantalum(V) (Ta(V)) compound, in which the molar ratio of chromium to tantalum is in a range of about 15:1 to about 1:1; and
(c) impregnating a porous, solid support material with the precursor solution to yield a bimetallic, supported catalyst for trimerization of ethylene having at least one Cr(III) catalytic metal and at least one Ta(V) catalytic metal.

11. A method of as recited in claim 10, further comprising:
washing the impregnated porous, solid support material with at least one solvent to remove unbound chromium and tantalum species;
removing the solvent to yield a bimetallic, supported catalyst for trimerization of ethylene.

12. A method of as recited in claim 11, the removing further comprising evaporating the solvent under vacuum.

13. A method of as recited in claim 10, the impregnating further comprising forming a chemical bond between the porous, solid support material and the at least one Cr(III) catalytic metal or the at least one Ta(V) catalytic metal.

14. A method of as recited in claim 13, wherein the chemical bond is a metal-oxygen bond via an oxygen that is bound to the support material.

15. A method as recited in claim 10, wherein the porous, solid support material is selected from the group consisting of silica, alumina, zeolite, activated carbon, at least one molecular sieve, and combinations thereof.

16. A method as recited in claim 15, wherein the porous, solid support material is calcined at a temperature in a range from about 100° C. to about 500° C. for about 10 minutes to about 5 hours prior to being impregnated with the at least one catalytic Cr(III) compound and the at least one catalytic Ta(V) compound.

17. A method as recited in claim 10, wherein the catalytic Cr(III) compound is selected from the group consisting of chromium chloride, chromium bromide, chromium fluoride, chromium nitrate, chromium sulfate, chromium phosphate, chromium acetate, chromium acetylacetonate, chromium 2-ethyl hexanoate, chromium bistrimethylsilylamido (Cr(NTMS₂)ₙ), derivatives thereof, and combinations thereof.

18. A method as recited in claim 10, wherein the catalytic Ta(V) compound is selected from the group consisting of tantalum chloride, tantalum bromide, tantalum fluoride, tantalum iodide, tantalum pentamethylcyclopentadienyl chloride (TaCp*Clₙ), tantalum dimethylamine (Ta(NMe₂)₅), Ta(NMe₂)₃Cl₂, Ta(NMe₂)₄Cl, tantalum hydrotris(pyrazolyl)borato chloride (TaTpCl₄), tantalum hydrotris(3,5-dimethylpyrazolyl)borato chloride (TaTp*Cl₄), derivatives thereof, and combinations thereof.

19. A method of as recited in claim 9, wherein the ratio of chromium to tantalum in the bimetallic, supported ethylene trimerization catalyst is in a range from about 5:1 to about 1:5.

20. A method of as recited in claim 9, wherein the ratio of chromium to tantalum in the bimetallic, supported ethylene trimerization catalyst is in a range from about 2:1 to about 1:2.

21. A method for catalytically producing 1-hexene from ethylene, comprising:
(a) providing a bimetallic, supported catalyst for trimerization of ethylene, the catalyst including:
a porous, solid support material;
at least one halogenated and/or organometallic chromium compound disposed on the porous, solid support material; and
at least one halogenated and/or organometallic tantalum compound disposed on the porous, solid support material;
(b) forming a reaction mixture in a reaction vessel, the reaction mixture including:
the bimetallic, supported catalyst and an organic solvent;
pressurized ethylene gas; and
a trialkyl-aluminum compound, 2,5-dimethylpyrrole, and hexachloroethane in amounts sufficient for catalysis; and
(c) reacting the reaction mixture to yield 1-hexene.

22. A method as recited in claim 21, the reacting further comprising maintaining a temperature in a range between about 50° C. and about 140° C. in the reaction vessel.

23. A method as recited in claim 22, wherein the temperature is in a range between about 90° C. and about 110° C.

24. A method as recited in claim 21, wherein the pressure of the ethylene gas is in a range from about 1 bar to about 100 bar.

25. A method as recited in claim 21, wherein the pressure of the ethylene gas is in a range from about 50 bar to about 70 bar.

26. A method of as recited in claim 21, wherein catalytic turnover is in a range from about 100 g 1-hexene/g metal/hr to about 5200 g hexene/g metal/hr.

27. A method of as recited in claim 21, wherein 1-hexene selectivity of the catalyst is at least 50%.

28. A method of as recited in claim 21, wherein 1-hexene selectivity of the catalyst is at least 70%.

29. A method of as recited in claim 21, wherein 1-hexene selectivity of the catalyst is at least 90%.

30. A method as recited in claim 21, further comprising reusing the catalyst in at least one subsequent reaction to yield 1-hexene.

31. A method as recited in claim 21, wherein the reaction mixture is substantially free of molecular oxygen.

32. A method as recited in claim 21, wherein the reaction mixture includes molecular oxygen.
